(19) United States
(12) Patent Application Publication
(10) Pub. No.: US 2012/0010292 A1
(43) Pub. Date: Jan. 12, 2012

(54) MODIFICATION I OF 4-(PHENYL)ETHYL]-AMINO}METHYL)BENZOIC ACID

(75) Inventors: Alfons Grunenberg, Dormagen (DE); Franz-Josef Mais, Duesseldorf (DE); Katharina Tenbieg, Dortmund (DE); Birgit Keil, Duesseldorf (DE)

(73) Assignee: BAYER SCHERING PHARMA AKTIENGESELLSCHAFT, Berlin (DE)

(21) Appl. No.: 13/132,529

(22) PCT Filed: Dec. 4, 2009
(86) PCT No.: PCT/EP2009/008664

§ 371 Date: Sep. 27, 2011

(30) Foreign Application Priority Data
Dec. 17, 2008 (DE) 10 2008 062 689.9

(51) Int. Cl. A61K 31/197; C07C 227/00; A61P 9/00; C07C 229/34

(52) U.S. Cl. 514/567; 562/444

(57) ABSTRACT

The invention relates to novel forms of 4-({(4-carboxybutyl)[2-(2-{[4-(2-phenyl-ethyl)benzyl]oxy}phenyl)ethyl]amino}methyl)benzoic acid, in particular to the modification I, to processes for their preparation, to medicaments comprising them and to their use for fighting diseases.

The invention relates to novel forms of 4-({(4-carboxybutyl)[2-(2-{[4-(2-phenylethyl)benzyl]oxy}phenyl)ethyl]amino}methyl)benzoic acid, in particular to the modification I, to processes for their preparation, to medicaments comprising them and to their use for fighting diseases.

4-({(4-Carboxybutyl)[2-(2-{[4-(2-phenylethyl)benzyl]oxy}phenyl)ethyl]amino}methyl)benzoic acid is described in WO 01/019780 and corresponds to the compound of the formula (I):

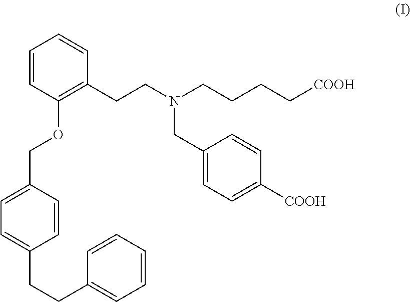

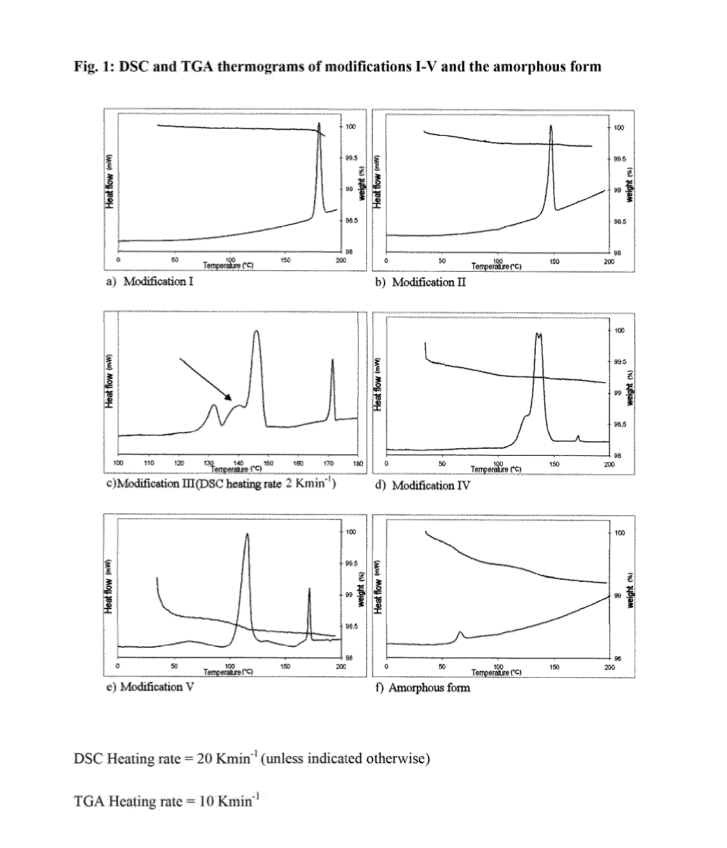

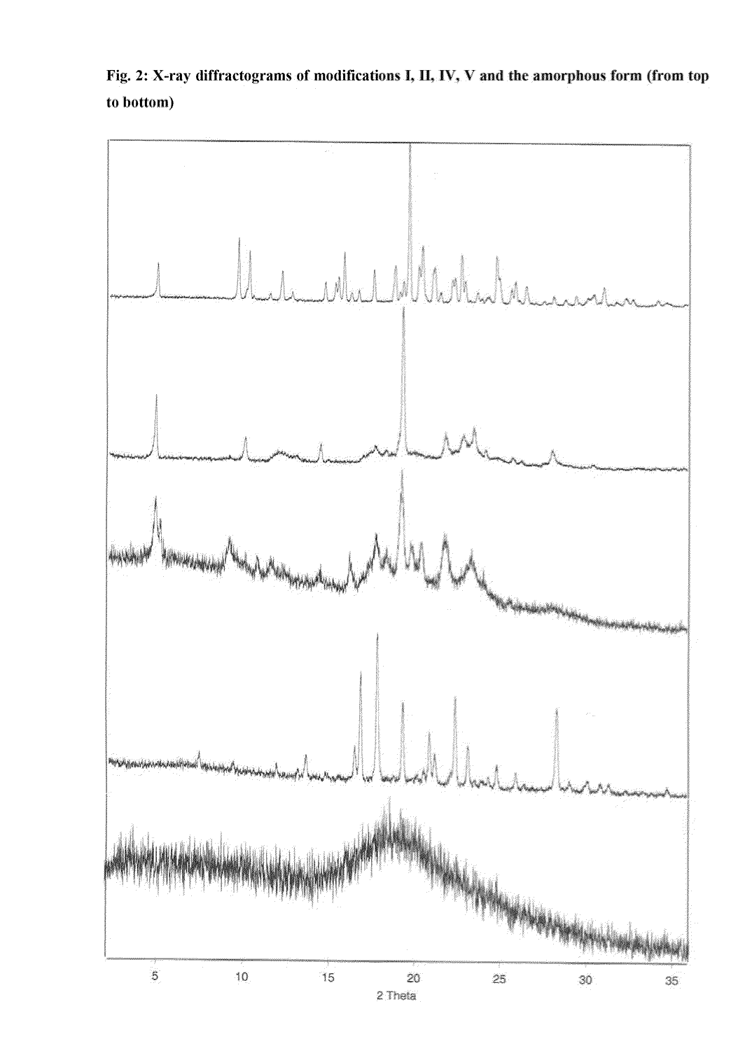

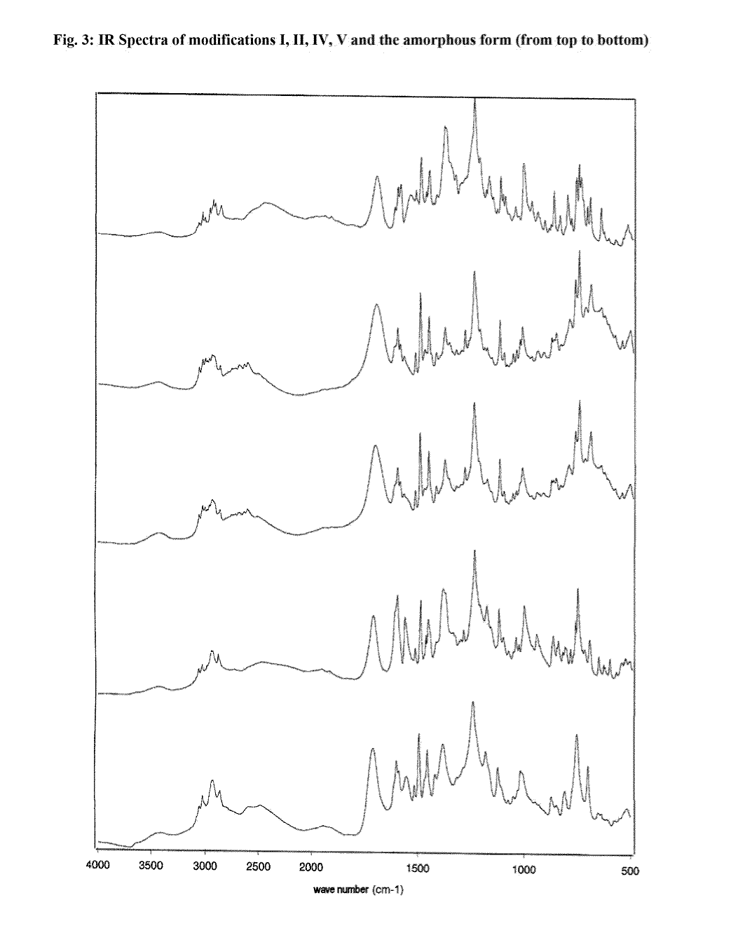

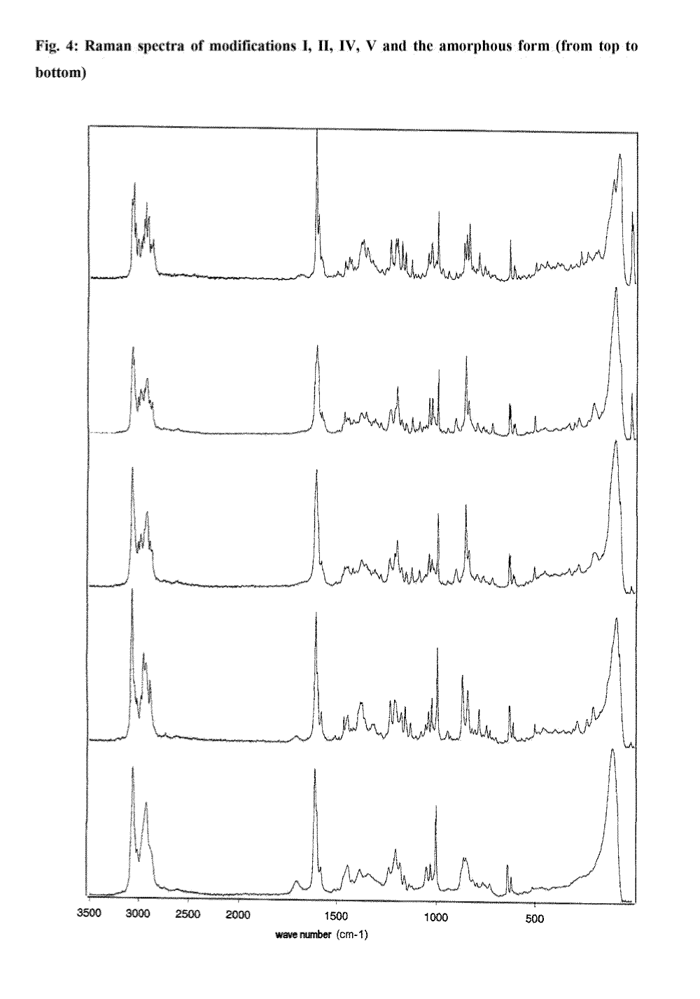

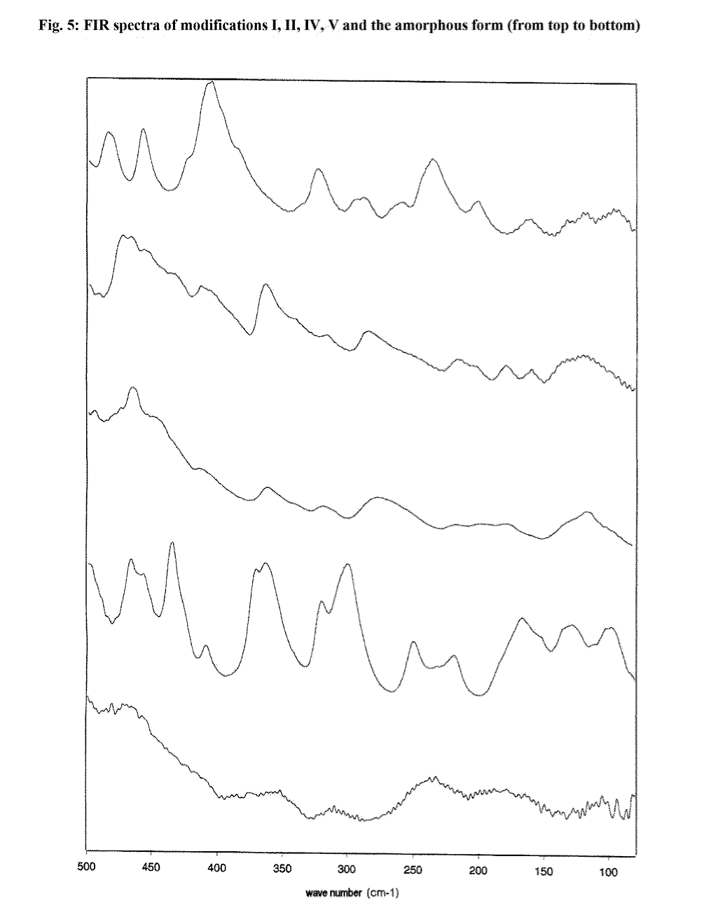

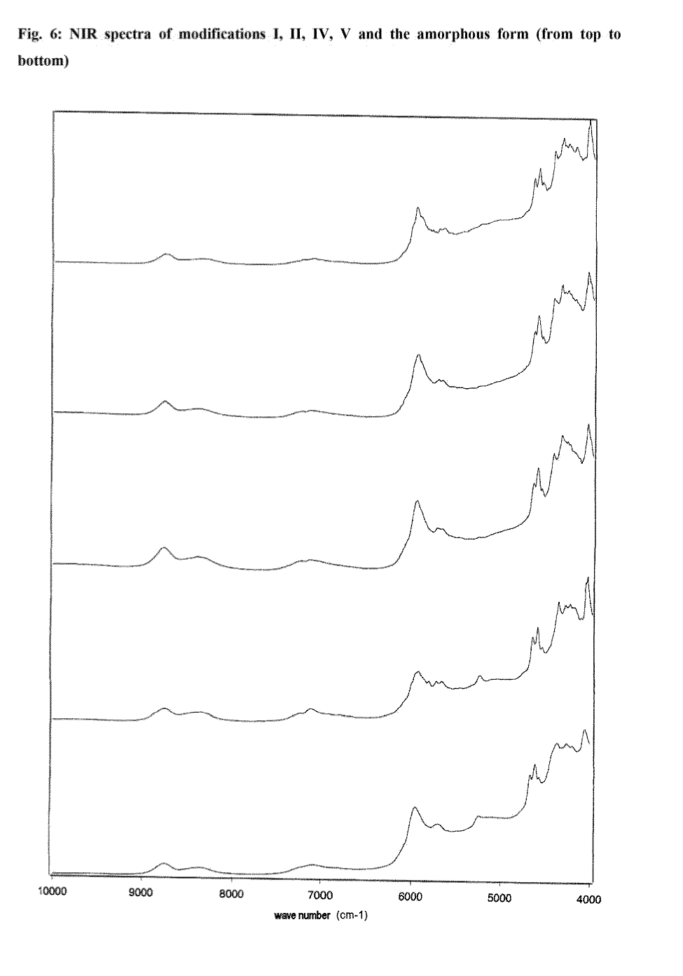

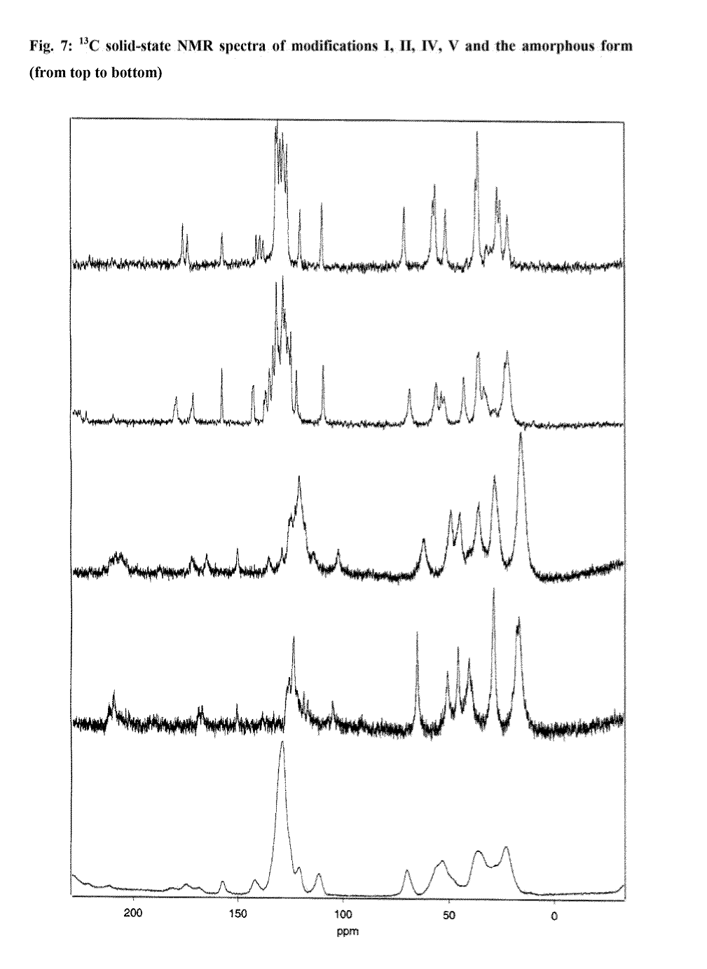

Preparation and use of the compound of the formula (I) for treating, for example, cardiovascular disorders are already known from WO 01/019780. Using the procedure described therein, the compound of the formula (I) is obtained in the form of a crystal modification which is referred to as modification IV hereinbelow. Modification IV has a melting point of 129° C. and a characteristic X-ray diffractogram, IR spectrum, Raman spectrum, FIR spectrum, NIR spectrum and ¹³C solid state NMR spectrum (Tab. 1-7, FIG. 1-7).

It has now been found that modification IV is metastable and thus not suitable for use in pharmaceutical formulations such as, for example, solid and semi-solid preparations.

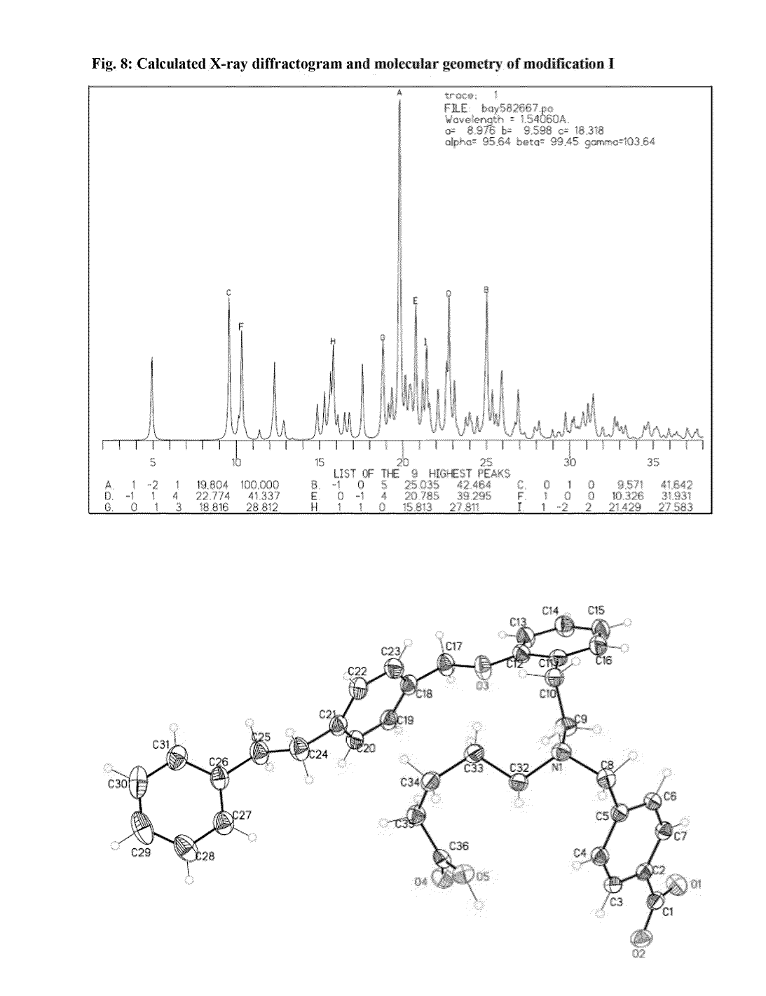

Surprisingly, four further polymorphic forms and the amorphous form have been found. Compared to modification IV, known from WO 01/019780, the polymorphic forms have markedly different melting points 170° C. (modification I), 142° C. (modification II), 135° C. (modification III) and 99° C. (modification V), and each of these modifications has a characteristic X-ray diffractogram, IR spectrum, Raman spectrum, FIR spectrum, NIR spectrum and ¹³C solid state NMR spectrum (Tab. 1-7, FIG. 1-7). In addition, FIG. 8 shows the crystal structure of the compound of the formula (I) in modification I.

Furthermore, surprisingly, we have found two polymorphic monohydrates A and B, a semihydrate, a methanol solvate and a methanol/water solvate of the compound of the formula (I). The monohydrates each contain a molecule of water, the semihydrate contains ½ molecule of water and the methanol solvate contains 1 molecule of methanol per compound of the formula (I). The methanol/water solvate is a mixed form of the isomorphic semihydrate and the methanol solvate. Each of the two polymorphic monohydrates A and B, the semihydrate, the methanol solvate and the methanol/water solvate of the compound of the formula (I) has a characteristic X-ray diffractogram, IR spectrum, Raman spectrum, FIR spectrum, NIR spectrum and ¹³C solid state NMR spectrum (Tab. 1-7). For the semihydrate and the methanol solvate, the crystal structure was determined (Tab. 8).

The present invention provides the compound of the formula (I) in modification I.

Surprisingly, the modification I of the compound of the formula (I) is thermodynamically stable and storage-stable even after processing to suspensions. By using, according to the invention, the compound of the formula (I) in the stable modification I, it is ensured that an unwanted conversion into another modification and concomitant changes in the properties of the compound of the formula (I), such as, for example, solubility or bioavailability, is prevented. This increases the safety and quality of preparations comprising the compound of the formula (I), and patient risk is reduced.

In the pharmaceutical formulations, the compound of the formula (I) in modification I according to the invention is employed in high purity. For reasons of stability, a pharmaceutical formulation comprises mainly the compound of the formula (I) in modification I and no major amounts of any other form of the compound of the formula (I). Preferably, the medicament comprises more than 90 percent by weight, particularly preferably more than 95 percent by weight, of the compound of the formula (I) in the modification I based on the total amount of the compound of the formula (I) present.

The present invention furthermore provides the use of the compound of the formula (I) in modification I for preparing a medicament for treating diseases, in particular for treating cardiovascular disorders.

The compound of the formula (I) in modification I brings about vasorelaxation and an inhibition of platelet aggregation and leads to a lowering of blood pressure and an increase in the coronary blood flow. These effects are mediated by direct stimulation of soluble guanylae cyclase and an intracellular increase in cGMP.

It can therefore be employed in medicaments for the treatment of cardiovascular disorders such as, for example, for the treatment of high blood pressure and heart failure, stable and unstable Angina pectoris, peripheral and cardiac vascular disorders, of arrhythmias, for the treatment of thromboembolic disorders and ischemias such as myocardial infarction, stroke, transistory and ischemic attacks, disturbances of peripheral blood flow, prevention of restenoses such as after thrombolysis therapies, percutaneous transluminal angioplasties (PTAs), percutaneous transluminal coronary angioplasties (PTCAs), bypass and for the treatment of arteriosclerosis, fibrotic disorders, such as fibrosis of the liver or pulmonary fibrosis, asthmatic disorders and diseases of the urogenital systems such as, for example, prostate hypertrophy, erectile dysfunction, female sexual dysfunction and incontinence and also for the treatment of glaucoma.

It can also be used for fighting diseases of the central nervous system characterized by disturbances of the NO/cGMP system. It is suitable in particular for removing cognitive deficits, for improving learning and memory performances and for treating Alzheimer's disease. It is also suitable for treating disorders of the central nervous system such as states of anxiety, tension and depression, CNS-related sexual dysfunctions and sleep disturbances, and for controlling pathological disturbances of the intake of food, stimulants and addictive substances.

It is furthermore also suitable for regulating cerebral blood flow and thus represents an effective agent for controlling migraine.

It is also suitable for the prophylaxis and control of the sequelae of cerebral infarction (apoplexia cerebri) such as stroke, cerebral ischemias and cranial cerebral trauma. It can likewise be employed for controlling states of pain.

In addition, it has an anti-inflammatory effect and can therefore be employed as an anti-inflammatory agent.

The present invention furthermore provides a method for treating disorders, in particular the disorders mentioned above, using an effective amount of the compound of the formula (I) in modification I.

The compound of the formula (I) in modification I can be administered in a suitable manner, such as, for example, orally, parenterally, pulmonarily, nasally, sublingually, lingually, buccally, rectally, dermally, transdermally, conjunctivally, optically, vaginally, as stents or as an implant.

For these administration routes, the compound according to the invention can be administered in suitable administration forms.

Suitable for oral administration are administration forms working according to the prior art, which release the compound of the formula (I) in modification I rapidly and/or in modified form, such as, for example, tablets (non-coated or coated tablets, for example coated with enteric, slowly dissolving or insoluble coats which control the release of the compound according to the invention), tablets which decompose rapidly in the oral cavity or films/wafers, films/lyophylisates, capsules (for example hard gelatin capsules or soft gelatin capsules), sugar-coated tablets, granules, pellets, powders, suspensions or aerosols.

Parenteral administration can take place with circumvention of an absorption step (for example intravenous, intraarterial, intracardiac, intraspinal or intralumbar) or with involvement of an absorption (for example intramuscular, subcutaneous, intracutaneous, percutaneous or intraperitoneal). For parenteral administration, suitable administration forms are, inter alia, injection and infusion preparations in the form of suspensions, lyophilizates or sterile powders.

Suitable for the other administration routes are, for example, pharmaceutical forms for inhalation (inter alia powder inhalers, nebulizers), tablets, films/wafers or capsules to be applied lingually, sublingually or buccally, suppositories, car or eye preparations, vaginal capsules, aqueous suspensions (lotions, shake lotions), lipophilic suspensions, ointments, creams, transdermal therapeutic systems (such as, for example, patches), pastes, dusting powders, implants or stents.

The compound according to the invention can be converted into the administration forms mentioned. This may take place in a manner known per se by mixing with inert non-toxic, pharmaceutically acceptable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and taste and/or odor corrigents.

The present invention furthermore provides medicaments comprising at least the compound of the formula (I) in modification I, usually together with one or more inert non-toxic, pharmaceutically suitable auxiliaries such as, for example, binders, fillers, etc., and their use for the purposes mentioned above.

In general, it has been found to be advantageous to administer the compound according to the invention in total amounts of from about 0.5 to about 500, preferably from 5 to 100, mg/kg of body weight per day, if appropriate in the form of a plurality of individual doses, to obtain the desired results. An individual dose contains the active compound in amounts of from about 1 to about 80, preferably 3 to 30, mg/kg of body weight.

The invention furthermore provides a process for preparing the compound of the formula (I) in modification IV, by suspending the compound of the formula (I), for example in modification IV, in an inert solvent and stirring or shaking at a temperature of from 10° C. to the reflux temperature of the solvent, preferably at from 15° C. to 35° C., particularly preferably at from 20 to 30° C., until the desired degree of conversion has been achieved, particularly preferably to quantitative conversion into modification I. The resulting crystals of modification I are separated off and, to remove the solvent present, dried at room temperature or at elevated temperature until the weight remains constant.

It is also possible to prepare the compound of the formula (I) in modification I by dissolving, for example, the compound of formula (I) in modification IV in an inert solvent and allowing the solution to stand at room temperature until the solvent has evaporated.

Preferably, the compound of the formula (I) in modification I is prepared by dissolving the compound of the formula (I) in modification IV in ethanol and allowing the solution to stand at room temperature until the compound of the formula (I) in modification I has crystallized out.

Suitable inert solvents are lower alcohols, such as methanol, ethanol, n-propanol, isopropanol, n-butanol, sec-butanol, iso-butanol, 1-pentanol, or ketones, such as acetone, or alkanes, such as n-pentane, cyclopentane, n-hexane, cyclohexane, or tetrahydrofuran, acetonitrile, toluene, ethyl acetate, 1,4-dioxane or mixtures of the solvents mentioned. Preference is given to acetonitrile, isopropanol, ethanol or mixtures of the solvents mentioned.

In general, the preparation processes are carried out under atmospheric pressure. However, it is also possible to operate under elevated or reduced pressure, for example at from 0.5 to 5 bar.

The percentages in the tests and examples below are, unless indicated otherwise, percentages by weight; parts are parts by weight. Solvent ratios, dilution ratios and concentrations of liquid/liquid solutions are in each case by volume.

### WORKING EXAMPLES

The DSC and TGA thermograms were obtained using a Differential Scanning calorimeter DSC 7 or Pyris-1 and a Thermogravimetric Analyzer TGA 7 from Perkin-Elmer. The X-ray diffractograms were recorded in a Stoe transmission diffractometer. The IR, FIR, NIR and Raman spectra were recorded using Fourier-IR Spectrometers IFS 66v (IR, FIR), IFS 28/N (NIR) and RFS 100 (Raman) from Bruker. The ¹³C solid state NMR spectra were recorded on a Bruker DRX 400.

### Example 1

### Preparation of 4-({(4-carboxybutyl)[2-(2-{[4-(2-phenylethyl)benzyl]oxy}phenyl)ethyl]amino}-methyl)benzoic acid in modification I

### Example 1.1

About 100 mg of 4-({(4-carboxybutyl)[2-(2-{[4-(2-phenylethyl)benzyl]oxy}phenyl)ethyl]amino}-methyl)benzoic acid in modification IV are suspended in 1 ml of ethyl acetate and shaken at 25° C. After one week, the suspension is filtered and the residue is dried at room temperature at ambient humidity. The residue is examined by thermoanalysis and corresponds to the title compound in modification I.

### Example 1.2

About 0.4 g of 4-({(4-carboxybutyl)[2-(2-{[4-(2-phenylethyl)benzyl]oxy}phenyl)ethyl]amino}-methyl)benzoic acid in modification IV are suspended in 5 ml of acetonitrile and stirred under reflux at 50° C. After one week, the suspension is filtered and the residue is dried at room temperature at ambient humidity. The residue is examined by X-ray diffractometry and corresponds to the title compound in modification I.

### Example 1.3

About 400 mg of 4-({(4-carboxybutyl)[2-(2-{[4-(2-phenylethyl)benzyl]oxy}phenyl)ethyl]amino}-methyl)benzoic acid in modification IV are dissolved in about 200 ml of hot ethanol and filtered. One fourth of the solution is allowed to stand at room temperature until the solvent has evaporated. The residue is examined by X-ray diffractometry and corresponds to the title compound in modification I.

### Example 1.4

About 0.3 g of 4-({(4-carboxybutyl)[2-(2-{[4-(2-phenylethyl)benzyl]oxy}phenyl)ethyl]amino}-methyl)benzoic acid in modification IV are suspended in 5 ml of isopropanol and stirred under reflux at 80° C. After 4 days, the suspension is filtered and the residue is dried at room temperature at ambient humidity. The residue is examined by X-ray diffractometry and corresponds to the title compound as modification I.

### Example 2

### Preparation of the monohydrate A of 4-({(4-carboxybutyl)[2-(2-{[4-(2-phenylethyl)benzyl]oxy}phenyl)ethyl]amino}methyl)benzoic acid

### Example 2.1

About 1 g of 4-({(4-carboxybutyl)[2-(2-{[4-(2-phenylethyl)benzyl]oxy}phenyl)ethyl]amino}-methyl)benzoic acid in modification IV are suspended in 40 ml of ethanol:water (1:1) and stirred at room temperature. After one week, the suspension is filtered and the residue is dried at room temperature at ambient humidity. The residue is examined by X-ray diffractometry and corresponds to the title compound as monohydrate A.

### Example 2.2

About 80 mg of 4-({(4-carboxybutyl)[2-(2-{[4-(2-phenylethyl)benzyl]oxy}phenyl)ethyl]amino}-methyl)benzoic acid in modification IV are suspended in 1 ml of tetrahydrofuran:water (1:3) and shaken at 25° C. After one week, the suspension is filtered and the residue is dried at room temperature at ambient humidity. The residue is examined by thermoanalysis and corresponds to the title compound as monohydrate A.

### Example 2.3

About 0.4 g of 4-({(4-carboxybutyl)[2-(2-{[4-(2-phenylethyl)benzyl]oxy}phenyl)ethyl]amino}-methyl)benzoic acid in modification IV are suspended, in 5 ml of ethanol:water (1:1) and stirred under reflux at 50° C. After one week, the suspension is filtered and the residue is dried at room temperature at ambient humidity. The residue is examined by X-ray diffractometry and corresponds to the title compound as monohydrate A.

### Example 2.4

About 80 mg of 4-({(4-carboxybutyl)[2-(2-{[4-(2-phenylethyl)benzyl]oxy}phenyl)ethyl]amino}-methyl)benzoic acid in modification IV are suspended in 1.5 ml of isopropanol:water (2:1) and shaken at 25° C. After one week, the suspension is filtered and the residue is dried at room temperature at ambient humidity. The residue is examined by thermoanalysis and corresponds to the title compound as monohydrate A.

### Example 2.5

About 0.3 g of 4-({(4-carboxybutyl)[2-(2-{[4-(2-phenylethyl)benzyl]oxy}phenyl)ethyl]amino}-methyl)benzoic acid in modification IV are suspended in 5 ml of ethanol:water (1:1) and stirred under reflux at 80° C. After 4 days, the suspension is filtered and the residue is dried at room temperature at ambient humidity. The residue is examined by X-ray diffractometry and corresponds to the title compound as monohydrate A.

### Example 3

### Preparation of the monohydrate B of 4-({(4-carboxybutyl)[2-(2-{[4-(2-phenylethyl)benzyl]oxy}phenyl)ethyl]amino}methyl)benzoic acid

### Example 3.1

About 0.4 g of 4-({(4-carboxybutyl)[2-(2-{[4-(2-phenylethyl)benzyl]oxy}phenyl)ethyl]amino}-methyl)benzoic acid in modification IV are dissolved in about 200 ml of hot acetone and filtered. One fourth of the solution is allowed to stand at room temperature until the solvent has evaporated. The residue is examined by X-ray diffractometry and corresponds to the title compound as monohydrate B.

### Example 3.2

About 0.4 g of 4-({(4-carboxybutyl)[2-(2-{[4-(2-phenylethyl)benzyl]oxy}phenyl)ethyl]amino}-methyl)benzoic acid in modification IV are dissolved in about 200 ml of hot isopropanol and filtered. Water is added to one fourth of the solution until the active compound precipitates. The precipitated active compound is isolated and dried at room temperature at ambient humidity. The active compound is examined by thermoanalysis and corresponds to the title compound as monohydrate B.

### Example 3.3

About 0.4 g of 4-({(4-carboxybutyl)[2-(2-{[4-(2-phenylethyl)benzyl]oxy}phenyl)ethyl]amino}-methyl)benzoic acid in modification IV are dissolved in about 200 ml of hot acetone and filtered. One fourth of the solution is allowed to stand at 5 to 8° C. until the solvent has evaporated. The residue is examined by thermoanalysis and corresponds to the title compound as monohydrate B.

### Example 3.4

About 0.4 g of 4-({(4-carboxybutyl)[2-(2-{[4-(2-phenylethyl)benzyl]oxy}phenyl)ethyl]amino}-methyl)benzoic acid in modification IV are dissolved in about 200 ml of hot acetonitrile and filtered. Water is added to one fourth of the solution until the active compound precipitates. The precipitated active compound is isolated and dried at room temperature at ambient humidity. The active compound is examined by X-ray diffractometry and corresponds to the title compound as monohydrate B.

### Example 4

### Preparation of 4-({(4-carboxybutyl)[2-(2-{[4-(2-phenylethyl)benzyl]oxy}phenyl)ethyl]amino}-methyl)benzoic acid in modification II

### Example 4.1

About 100 mg of the monohydrate A of 4-({(4-carboxybutyl)[2-(2-{[4-(2-phenylethyl)benzyl]oxy}phenyl)ethyl]amino}-methyl)benzoic acid are tempered at 70° C. in a drying cabinet for 10 min. The active compound is examined by X-ray diffractometry and corresponds to the title compound in modification II.

### Example 4.2

About 100 mg, of the monohydrate A of 4-({(4-carboxybutyl)[2-(2-{[4-(2-phenylethyl)benzyl]oxy}phenyl)ethyl]amino}methyl)benzoic acid are tempered at 100° C. in a drying cabinet for 5 min. The active compound is examined by thermoanalysis and corresponds to the title compound in modification II.

### Example 4.3

About 100 mg, of the monohydrate A of 4-({(4-carboxybutyl)[2-(2-{[4-(2-phenylethyl)benzyl]oxy}phenyl)ethyl]amino}methyl)benzoic acid are stored over phosphorus pentoxide at room temperature for 2 days. The active compound is examined by thermoanalysis and corresponds to the title compound in modification II.

### Example 5

### Preparation of the semihydrate of 4-({(4-carboxybutyl)[2-(2-{[4-(2-phenylethyl)benzyl]oxy}phenyl)ethyl]amino}methyl)benzoic acid

### Example 5.1

About 0.4 g of 4-({(4-carboxybutyl)[2-(2-{[4-(2-phenylethyl)benzyl]oxy}phenyl)ethyl]amino}-methyl)benzoic acid in modification II are dissolved in about 200 ml of hot methanol and filtered. One fourth of the solution is allowed to stand at about −20° C. until the solvent has evaporated. The crystal structure of the residue is determined. The residue corresponds to the title compound as semihydrate.

### Example 6

### Preparation of the methanol solvate of 4-({(4-carboxybutyl)[2-(2-{[4-(2-phenylethyl)benzyl]oxy}phenyl)ethyl]amino}methyl)benzoic acid

### Example 6.1

4 g of 4-({(4-carboxybutyl)[2-(2-{[4-(2-phenylethyl)benzyl]oxy}phenyl)ethyl]amino}-methyl)benzoic acid in modification IV are dissolved in 1.5 l of hot methanol and filtered. The solutions are divided into five parts, allowed to stand at room temperature until the solvent has evaporated and combined to one sample. The crystal structure of the residue is determined. The residue corresponds to the title compound as methanol solvate.

### Example 7

### Preparation of the methanol/water solvate of 4-({(4-carboxybutyl)[2-(2-{[4-(2-phenylethyl)benzyl]oxy}phenyl)ethyl]amino}methyl)benzoic acid

### Example 7.1

About 0.6 g of 4-({(4-carboxybutyl)[2-(2-{[4-(2-phenylethyl)benzyl]oxy}phenyl)ethyl]amino}-methyl)benzoic acid in modification IV are dissolved in about 500 ml of hot methanol and filtered. The solution is allowed to stand at room temperature until the solvent has evaporated. The residue is examined by X-ray diffractometry and corresponds to the title compound as methanol/water solvate.

### Example 8

### Preparation of 4-({(4-carboxybutyl)[2-(2-{[4-(2-phenylethyl)benzyl]oxy}phenyl)ethyl]amino}-methyl)benzoic acid in modification III

### Example 8.1

2-3 mg of 4-({(4-carboxybutyl)[2-(2-{[4-(2-phenylethyl)benzyl]oxy}phenyl)ethyl]amino}-methyl)benzoic acid in modification. IV are heated in a DSC calorimeter. From the melt of modification IV, modification III crystallizes on further heating.

### Example 9

### Preparation of 4-({(4-carboxybutyl)[2-(2-{[4-(2-phenylethyl)benzyl]oxy}phenyl)ethyl]amino}-methyl)benzoic acid in modification V

### Example 9.1

About 100 mg of the methanol/water solvate of 4-({(4-carboxybutyl)[2-(2-{[4-(2-phenylethyl)benzyl]oxy}phenyl)ethyl]amino}methyl)benzoic acid are tempered at 70° C. in a drying cabinet for 3 h. The active compound is examined by thermoanalysis and corresponds to the title compound in modification V.

### Example 9.2

About 70 mg of the methanol/water solvate of 4-({(4-carboxybutyl)[2-(2-{[4-(2-phenylethyl)benzyl]oxy}phenyl)ethyl]amino}methyl)benzoic acid are stored over phosphorus pentoxide at room temperature for 2 days. The active compound is examined by, X-ray diffractometry and corresponds to the title compound in modification V.

### Example 10

### Preparation of the amorphous form of 4-({(4-carboxybutyl)[2-(2-{[4-(2-phenylethyl)benzyl]oxy}phenyl)ethyl]amino}-methyl)benzoic acid

### Example 10.1

About 80 mg of 4-({(4-carboxybutyl)[2-(2-{[4-(2-phenylethyl)benzyl]oxy}phenyl)ethyl]amino}-methyl)benzoic acid in Modification IV are melted on a Kofler hot stage and rapidly cooled to room temperature. The active compound is examined by X-ray diffractometry and corresponds, to the title compound in amorphous form.

### Example 10.2

About 100 mg of 4-({(4-carboxybutyl)[2-(2-{[4-(2-phenylethyl)benzyl]oxy}phenyl)ethyl]amino}-methyl)benzoic acid in modification IV are melted at 180° C. in a drying cabinet for 10 min and rapidly cooled to room temperature. The active compound is examined by X-ray diffractometry and corresponds to the title compound in amorphous form.

TABLE 1
Differential Scanning Calorimetry and Thermogravimetry

| | | Loss of mass |
|---|---|---|
| | Melting point [° C.] | [% by weight] |
| Modification I | 170 | ≦0.2 |
| Modification II | 142 | ≦0.3 |
| Modification III | 135 | — |
| Modification IV | 129 | ≦1 |
| Modification V | 99 | ≦2 |
| Amorphous form | 51-61 (Glass | ≦1 |
| | transition) | |
| Monohydrate A | — | 3.1 |
| Monohydrate B | — | 3.1 |
| Methanol/water solvate | — | 1.6-5.4 depending on the |
| | | composition |

TABLE 2
X-ray diffractometry

| Modi- | Modi- | Modi- | Modi- | | | Methanol/ |
|---|---|---|---|---|---|---|
| fication | fication | fication | fication | Mono- | Mono- | water |
| I | II | IV | V | hydrate A | hydrate B | solvate |
| 4.9 | 4.8 | 4.8 | 7.3 | 5.1 | 5.0 | 6.7 |
| 9.6 | 10.0 | 5.0 | 9.4 | 9.1 | 9.5 | 7.2 |
| 10.2 | 11.9 | 9.1 | 11.9 | 10.1 | 10.7 | 9.3 |
| 10.5 | 14.4 | 10.7 | 13.1 | 11.0 | 15.2 | 12.1 |
| 11.4 | 17.6 | 11.5 | 13.6 | 12.8 | 16.2 | 12.9 |
| 12.1 | 18.3 | 14.4 | 14.7 | 13.2 | 17.6 | 13.4 |
| 12.7 | 19.3 | 16.1 | 16.5 | 14.5 | 17.8 | 16.2 |
| 14.7 | 21.8 | 17.6 | 16.8 | 15.4 | 19.8 | 16.5 |
| 15.3 | 22.9 | 18.3 | 17.8 | 16.0 | 20.4 | 17.5 |
| 15.4 | 23.4 | 19.2 | 19.3 | 16.7 | 21.7 | 17.8 |
| 15.8 | 24.2 | 19.7 | 20.2 | 17.6 | | 19.1 |
| 16.2 | 25.7 | 20.4 | 20.5 | 18.4 | | 20.5 |
| 16.6 | 28.1 | 21.7 | 20.9 | 18.8 | | 20.8 |
| 17.5 | | 23.3 | 21.2 | 19.3 | | 21.2 |
| 18.8 | | 24.0 | 22.4 | 19.9 | | 22.0 |
| 19.1 | | | 23.1 | 20.6 | | 22.8 |
| 19.3 | | | 24.3 | 21.8 | | 23.4 |
| 19.6 | | | 24.9 | 22.4 | | 24.0 |
| 20.2 | | | 25.9 | 23.1 | | 24.4 |
| 20.4 | | | 28.3 | 24.0 | | 25.6 |
| 21.1 | | | 29.1 | 25.9 | | 27.8 |
| 21.5 | | | 30.1 | 27.3 | | 29.5 |
| 22.2 | | | 30.9 | 29.7 | | 30.3 |
| 22.3 | | | 34.8 | | | |
| 22.7 | | | | | | |
| 22.9 | | | | | | |
| 23.6 | | | | | | |
| 24.3 | | | | | | |
| 24.7 | | | | | | |
| 25.6 | | | | | | |
| 25.9 | | | | | | |
| 26.5 | | | | | | |
| 27.5 | | | | | | |
| 28.1 | | | | | | |
| 28.8 | | | | | | |
| 29.4 | | | | | | |
| 30.5 | | | | | | |
| 31.1 | | | | | | |
| 31.8 | | | | | | |
| 32.4 | | | | | | |
| 32.8 | | | | | | |
| 34.2 | | | | | | |
| 34.8 | | | | | | |
| 36.8 | | | | | | |

TABLE 3
IR Spectroscopy

| | | | | | | | Methanol/ |
|---|---|---|---|---|---|---|---|
| Modification | Modification | Modification | Modification | amorphous | Mono- | Mono- | water |
| I | II | IV | V | form | hydrate A | hydrate B | solvate |
| 3437 | 3431 | 3424 | 3421 | 3408 | 3396 | 3397 | 3420 |
| 3061 | 3058 | 3059 | 3060 | 3058 | 3058 | 3059 | 3060 |
| 3028 | 3026 | 3026 | 3027 | 3025 | 3026 | 3027 | 3028 |
| 3004 | 3000 | 3003 | 2941 | 2928 | 2941 | 2940 | 2941 |
| 2961 | 2983 | 2961 | 2928 | 2859 | 2863 | 2861 | 2926 |
| 2929 | 2962 | 2940 | 2875 | 1710 | 2663 | 2660 | 2872 |
| 2908 | 2940 | 2860 | 2727 | 1601 | 2606 | 2604 | 1714 |
| 2857 | 2860 | 2719 | 1711 | 1590 | 1704 | 2529 | 1611 |
| 2451 | 2719 | 2686 | 1600 | 1554 | 1602 | 1706 | 1601 |
| 1699 | 2684 | 2639 | 1565 | 1516 | 1589 | 1602 | 1565 |
| 1614 | 2638 | 2608 | 1517 | 1494 | 1561 | 1590 | 1517 |
| 1601 | 2607 | 1703 | 1490 | 1454 | 1518 | 1558 | 1491 |
| 1588 | 1700 | 1602 | 1466 | 1417 | 1495 | 1517 | 1466 |
| 1542 | 1602 | 1589 | 1454 | 1380 | 1478 | 1495 | 1455 |
| 1516 | 1590 | 1572 | 1383 | 1314 | 1467 | 1478 | 1417 |
| 1492 | 1572 | 1518 | 1339 | 1241 | 1454 | 1467 | 1372 |
| 1467 | 1518 | 1495 | 1302 | 1180 | 1419 | 1454 | 1335 |
| 1453 | 1495 | 1474 | 1289 | 1122 | 1380 | 1419 | 1314 |
| 1432 | 1474 | 1454 | 1239 | 1074 | 1339 | 1378 | 1303 |
| 1418 | 1455 | 1419 | 1182 | 1050 | 1323 | 1339 | 1289 |
| 1381 | 1419 | 1379 | 1163 | 1030 | 1297 | 1319 | 1283 |
| 1330 | 1380 | 1324 | 1124 | 1017 | 1284 | 1297 | 1239 |
| 1241 | 1359 | 1286 | 1105 | 947 | 1240 | 1284 | 1199 |
| 1217 | 1325 | 1242 | 1081 | 872 | 1217 | 1242 | 1183 |
| 1188 | 1300 | 1182 | 1046 | 848 | 1191 | 1218 | 1124 |
| 1174 | 1286 | 1123 | 1032 | 809 | 1182 | 1191 | 1106 |
| 1155 | 1243 | 1103 | 1007 | 752 | 1122 | 1181 | 1079 |
| 1135 | 1216 | 1091 | 950 | 699 | 1104 | 1122 | 1047 |
| 1120 | 1195 | 1062 | 872 | 652 | 1066 | 1104 | 1030 |
| 1109 | 1183 | 1046 | 855 | 637 | 1047 | 1066 | 1021 |
| 1098 | 1161 | 1018 | 848 | 613 | 1032 | 1047 | 1007 |
| 1051 | 1124 | 975 | 827 | 574 | 1013 | 1032 | 948 |
| 1012 | 1102 | 949 | 817 | 516 | 976 | 1017 | 873 |
| 976 | 1091 | 920 | 793 | | 949 | 976 | 849 |
| 948 | 1061 | 882 | 768 | | 940 | 930 | 827 |
| 915 | 1045 | 873 | 759 | | 931 | 875 | 811 |
| 896 | 1029 | 860 | 737 | | 883 | 861 | 795 |
| 885 | 1019 | 839 | 726 | | 876 | 845 | 781 |
| 872 | 974 | 802 | 703 | | 861 | 802 | 760 |
| 842 | 946 | 770 | 660 | | 846 | 777 | 703 |
| 809 | 919 | 752 | 636 | | 803 | 771 | 659 |
| 789 | 883 | 724 | 630 | | 778 | 751 | 637 |
| 767 | 872 | 698 | 608 | | 771 | 700 | 609 |
| 755 | 861 | 650 | 577 | | 751 | 650 | 578 |
| 745 | 839 | 634 | 554 | | 730 | 615 | 553 |
| 716 | 799 | 585 | 536 | | 708 | 585 | 527 |
| 702 | 771 | 552 | 516 | | 701 | 541 | |
| 651 | 754 | 515 | | | 651 | 518 | |
| 636 | 723 | | | | 615 | | |
| 615 | 697 | | | | 585 | | |
| 584 | 649 | | | | 543 | | |
| 527 | 635 | | | | 514 | | |
| | 618 | | | | | | |
| | 586 | | | | | | |
| | 553 | | | | | | |
| | 515 | | | | | | |

TABLE 4
Raman spectroscopy

| | | | | | | | Methanol/ |
|---|---|---|---|---|---|---|---|
| Modification | Modification | Modification | Modification | amorphous | Mono- | Mono- | water |
| I | II | IV | V | form | hydrate A | hydrate B | solvate |
| 3074 | 3062 | 3064 | 3061 | 3062 | 3061 | 3062 | 3060 |
| 3053 | 3052 | 3003 | 3015 | 3013 | 3008 | 3007 | 3015 |
| 3037 | 3000 | 2981 | 2974 | 2925 | 2978 | 2978 | 2938 |
| 3010 | 2981 | 2913 | 2942 | 1708 | 2924 | 2926 | 2875 |
| 2977 | 2942 | 2884 | 2916 | 1614 | 2879 | 2879 | 2834 |
| 2961 | 2915 | 2861 | 2876 | 1584 | 1611 | 1612 | 2725 |
| 2946 | 2886 | 1612 | 1713 | 1443 | 1588 | 1583 | 1713 |
| 2930 | 2861 | 1584 | 1612 | 1382 | 1582 | 1467 | 1613 |
| 2904 | 1611 | 1508 | 1584 | 1345 | 1510 | 1450 | 1602 |
| 2880 | 1584 | 1465 | 1509 | 1241 | 1474 | 1439 | 1583 |
| 2858 | 1497 | 1446 | 1490 | 1205 | 1449 | 1387 | 1467 |
| 1616 | 1466 | 1422 | 1467 | 1183 | 1439 | 1355 | 1445 |
| 1602 | 1454 | 1379 | 1447 | 1162 | 1388 | 1316 | 1373 |
| 1506 | 1444 | 1316 | 1430 | 1139 | 1356 | 1242 | 1334 |
| 1467 | 1421 | 1286 | 1419 | 1052 | 1336 | 1216 | 1285 |
| 1446 | 1382 | 1243 | 1383 | 1032 | 1316 | 1205 | 1240 |
| 1435 | 1359 | 1216 | 1318 | 1003 | 1287 | 1184 | 1211 |
| 1373 | 1316 | 1205 | 1239 | 862 | 1237 | 1161 | 1183 |
| 1355 | 1288 | 1182 | 1216 | 851 | 1216 | 1135 | 1161 |
| 1240 | 1239 | 1160 | 1183 | 765 | 1207 | 1093 | 1132 |
| 1213 | 1205 | 1131 | 1164 | 730 | 1184 | 1051 | 1082 |
| 1205 | 1184 | 1093 | 1137 | 642 | 1161 | 1033 | 1048 |
| 1182 | 1163 | 1062 | 1108 | 622 | 1135 | 1002 | 1031 |
| 1165 | 1131 | 1047 | 1084 | 108 | 1094 | 916 | 1003 |
| 1135 | 1112 | 1032 | 1062 | | 1050 | 861 | 946 |
| 1119 | 1094 | 1002 | 1048 | | 1033 | 849 | 873 |
| 1085 | 1074 | 952 | 1031 | | 1015 | 819 | 850 |
| 1053 | 1062 | 910 | 1003 | | 1002 | 800 | 795 |
| 1034 | 1046 | 862 | 951 | | 933 | 771 | 752 |
| 1014 | 1031 | 846 | 936 | | 912 | 727 | 736 |
| 1003 | 1002 | 804 | 876 | | 877 | 642 | 641 |
| 979 | 990 | 771 | 849 | | 862 | 636 | 621 |
| 950 | 954 | 727 | 828 | | 849 | 621 | 512 |
| 914 | 913 | 643 | 812 | | 818 | 515 | 468 |
| 871 | 863 | 637 | 792 | | 800 | 463 | 293 |
| 858 | 847 | 622 | 754 | | 771 | 396 | 251 |
| 844 | 803 | 615 | 737 | | 760 | 350 | 216 |
| 829 | 773 | 515 | 706 | | 726 | 310 | 98 |
| 796 | 759 | 465 | 642 | | 643 | 228 | 30 |
| 768 | 727 | 342 | 638 | | 636 | 108 | |
| 752 | 644 | 296 | 621 | | 619 | 85 | |
| 723 | 638 | 217 | 578 | | 516 | 29 | |
| 641 | 621 | 109 | 512 | | 458 | 21 | |
| 621 | 615 | 86 | 473 | | 388 | | |
| 601 | 515 | 29 | 407 | | 337 | | |
| 551 | 465 | | 369 | | 309 | | |
| 512 | 409 | | 342 | | 227 | | |
| 489 | 344 | | 301 | | 201 | | |
| 459 | 317 | | 251 | | 101 | | |
| 406 | 295 | | 219 | | 29 | | |
| 340 | 219 | | 103 | | | | |
| 286 | 111 | | 86 | | | | |
| 252 | 29 | | 29 | | | | |
| 197 | | | | | | | |
| 121 | | | | | | | |
| 92 | | | | | | | |
| 29 | | | | | | | |
| 23 | | | | | | | |

TABLE 5
FIR spectroscopy

| Modi- | Modi- | Modi- | Modi- | | | Methanol/ |
|---|---|---|---|---|---|---|
| fication | fication | fication | fication | Mono- | Mono- | water |
| I | II | IV | V | hydrate A | hydrate B | solvate |
| 485 | 492 | 495 | 466 | 497 | 464 | 482 |
| 458 | 473 | 486 | 457 | 491 | 446 | 465 |
| 405 | 467 | 475 | 435 | 484 | 321 | 431 |
| 324 | 457 | 466 | 409 | 464 | 277 | 409 |
| 289 | 413 | 450 | 371 | 446 | | 370 |
| 260 | 364 | 414 | 364 | 370 | | 351 |
| 236 | 317 | 363 | 321 | 320 | | 320 |
| 201 | 285 | 320 | 300 | 277 | | 297 |
| 161 | 217 | 278 | 250 | | | 248 |
| 120 | 179 | 218 | 233 | | | 229 |
| 96 | 160 | 199 | 220 | | | |
| | | 179 | 167 | | | |
| | | 118 | 128 | | | |
| | | | 110 | | | |
| | | | 102 | | | |
| | | | 98 | | | |

TABLE 6
NIR spectroscopy

| Modification | Modification | Modification | Modification | amorphous | Mono- | Mono- | Methanol/water |
|---|---|---|---|---|---|---|---|
| I | II | IV | V | form | hydrate A | hydrate B | solvate |
| 8767 | 8753 | 8759 | 8748 | 8750 | 8763 | 8756 | 8757 |
| 8371 | 8382 | 8391 | 8347 | 8358 | 8345 | 8347 | 8338 |
| 7101 | 7137 | 7133 | 7124 | 7091 | 7243 | 5974 | 7141 |
| 5974 | 5954 | 5957 | 5936 | 5956 | 6444 | 5710 | 5943 |
| 5724 | 5727 | 5740 | 5824 | 5697 | 5974 | 5031 | 5824 |
| 5669 | 5684 | 4665 | 5740 | 5243 | 5678 | 4666 | 5677 |
| 4669 | 4662 | 4616 | 5678 | 4668 | 5032 | 4614 | 5266 |
| 4613 | 4618 | 4572 | 5255 | 4613 | 4664 | 4571 | 4669 |
| 4576 | 4569 | 4442 | 4668 | 4570 | 4615 | 4377 | 4614 |
| 4439 | 4442 | 4350 | 4613 | 4363 | 4568 | 4351 | 4559 |
| 4343 | 4354 | 4288 | 4560 | 4259 | 4439 | 4311 | 4379 |
| 4279 | 4320 | 4064 | 4378 | 4197 | 4378 | 4255 | 4301 |
| 4197 | 4286 | | 4299 | 4057 | 4354 | 4193 | 4248 |
| 4053 | 4200 | | 4249 | | 4311 | 4150 | 4197 |
| | 4066 | | 4129 | | 4252 | 4062 | 4054 |
| | | | 4072 | | 4193 | | |
| | | | 4054 | | 4064 | | |

TABLE 7
¹³C Solid state NMR spectroscopy

| | | | | | | | Methanol/ |
|---|---|---|---|---|---|---|---|
| Modification | Modification | Modification | Modification | amorphous | Mono- | Mono- | water |
| I | II | IV | V | form | hydrate A | hydrate B | solvate |
| 176 | 179 | 172 | 167 | 157 | 172 | 172 | 188 |
| 174 | 171 | 165 | 150 | 142 | 167 | 166 | 169 |
| 158 | 158 | 151 | 139 | 129 | 151 | 150 | 165 |
| 142 | 143 | 136 | 133 | 121 | 138 | 136 | 150 |
| 140 | 137 | 129 | 124 | 111 | 136 | 133 | 136 |
| 138 | 135 | 125 | 119 | 70 | 133 | 130 | 133 |
| 132 | 134 | 121 | 117 | 53 | 130 | 126 | 127 |
| 131 | 132 | 114 | 105 | 36 | 126 | 122 | 126 |
| 130 | 129 | 103 | 65 | 23 | 125 | 120 | 124 |
| 129 | 128 | 62 | 51 | | 123 | 118 | 120 |
| 127 | 127 | 49 | 46 | | 119 | 114 | 117 |
| 121 | 125 | 44 | 40 | | 115 | 104 | 115 |
| 110 | 123 | 36 | 29 | | 105 | 64 | 103 |
| 71 | 110 | 28 | 17 | | 64 | 50 | 64 |
| 57 | 69 | 15 | | | 50 | 47 | 49 |
| 51 | 56 | | | | 47 | 36 | 46 |
| 36 | 54 | | | | 36 | 29 | 41 |
| 27 | 43 | | | | 31 | 16 | 39 |
| 26 | 36 | | | | 29 | | 28 |
| 22 | 33 | | | | 16 | | 16 |
| | 22 | | | | | | |

TABLE 8
Crystal structure data

| | | | Monomethanol |
|---|---|---|---|
| | Modification 1 | Semihydrate | solvate |
| Temperature [K] | 100 | 100 | 90 |
| Crystal system | triclinic | orthorhombic | orthorhombic |
| Space group | P1 | P2₁2₁2₁ | P2₁2₁2₁ |
| Molecules per unit cell | 2 | 4 | 4 |
| Length of axis a [Å] | 8.9765(4) | 8.89810(10) | 9.1457(9) |
| Length of axis b [Å] | 9.5982(10) | 13.2961(2) | 13.4840(13) |
| Length of axis c [Å] | 18.318(2) | 26.0435(5) | 25.655(2) |
| α [°] | 95.636(9) | 90 | 90 |
| β [°] | 99.451(10) | 90 | 90 |
| γ [°] | 103.645(10) | 90 | 90 |
| Calculated density | 1.255 | 1.237 | 1.255 |
| [g cm⁻³] | | | |

The invention claimed is:

1. A compound of formula (I) 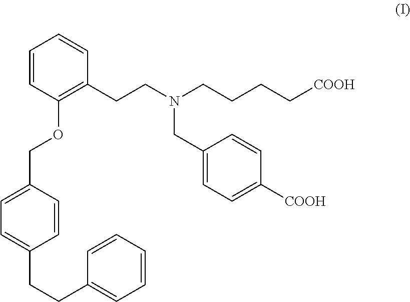
in modification I.

2. The compound of claim 1, characterized in that the X-ray diffractogram of the compound shows a peak maximum of the 2 theta angle at 9.6.

3. The compound of claim 1, characterized in that the X-ray diffractogram of the compound shows peaks of the 2 theta angle at 9.6, 15.8 and 24.7.

4. The compound of claim 1, characterized in that the Raman spectrum of the compound shows a band at 2930 cm⁻¹.

5. The compound of claim 1, characterized in that the Raman spectrum of the compound shows bands at 2930, 1616 and 1602 cm⁻¹.

6. The compound of claim 1, characterized in that the NIR spectrum of the compound shows bands at 5974, 4613 and 4053 cm⁻¹.

7. (canceled)

8. A pharmaceutical composition comprising a compound a of claim 1 and no major amounts of any other form of compound of formula (I).

9. A pharmaceutical composition comprising a compound of formula (I): 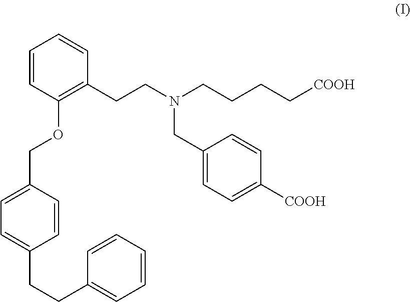
wherein the compound of claim 1 is present in an amount of more than 90 percent by weight, based on the total amount of the compound of formula (I) comprised therein.

10. A process for preparing the compound of claim 1, comprising suspending a compound of formula (I): 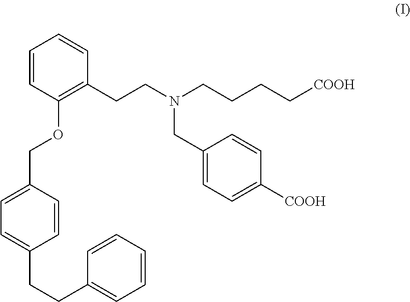
in modification IV in an inert solvent, and stirring or shaking at a temperature of from 10° C. to the reflux temperature of the solvent until quantitative conversion into modification I has been achieved.

11. (canceled)

12. A method of treatment of a cardiovascular disorders by administering to a patient in need thereof an effective amount of a compound of claim 1.
